(19) United States
(12) Patent Application Publication
(10) Pub. No.: US 2003/0078538 A1
(43) Pub. Date: Apr. 24, 2003

(54) Inflation device for dual balloon catheter

(76) Inventors: Paul V. Neale, San Diego, CA (US); John E. Henckel, Houston, TX (US); Kelly A. Pike, Half Moon Bay, CA (US); Matthew J. Wioncek, Sunnyvale, CA (US); Chris Andrews, Murrieta, CA (US)

Correspondence Address: BLAKELY, SOKOLOFF, TAYLOR & ZAFMAN LLP, Seventh Floor, 12400 Wilshire Boulevard, Los Angeles, CA, 90025-1026, US

(21) Appl. No.: 09/751,498

(22) Filed: Dec. 28, 2000

(51) Int. Cl. A61M 29/00

(52) U.S. Cl. 604/98.01; 604/101.02

(57) ABSTRACT

A balloon catheter inflation system. And inflation device includes a housing defining a chamber for an inflation medium. The housing can be coupled to a balloon catheter that has two balloons. The housing can be coupled to the catheter such that the chamber is in fluid communication with the balloons. The inflation system further includes a valve configuration operatively associated with the chamber to provide a first pressure to one of the balloons and a second pressure to the other balloon by a single inflating action that moves the inflation medium from the chamber to the balloons. The balloons of a multiple balloon catheter can each be inflated to a different pressure substantially simultaneously by a single inflating action.

### FIELD OF THE INVENTION

The present invention relates generally to the field of devices associated with surgical procedures that employ balloon catheters. More particularly, this invention relates to inflation devices for balloon catheters.

### BACKGROUND OF THE INVENTION

Balloon catheter systems are commonly used in arterial intervention procedures such as coronary angioplasty. For percutaneous transluminal coronary angioplasty (PTCA), a balloon catheter is typically used with a guide wire that is inserted into the patient's arteries. The guide wire can be inserted through a guiding catheter, which was previously introduced into the patient's arteries. The guide wire is inserted until its distal end is advanced past the diseased or stenotic area of the vessel, where there is a buildup of material. Balloon catheters typically have a guide wire lumen so that the proximal end of the guide wire is inserted into the distal end of the balloon catheter. The balloon catheter is then advanced over the guide wire until the balloon is adjacent the buildup of material. The balloon is then inflated to compress the buildup. Finally, the balloon is deflated and the catheter is pulled back up the guide wire and removed from the patient's artery. Restenosis of the artery often occurs after this procedure. Restenosis is when the same area of the vessel collapses or becomes clogged again.

Placing a stent at the area of the stenotic lesion can minimize the effect of restenosis. A stent is typically a tubular supporting structure made up of interlinked or intersected expandable tubular members, however, many different types of stents are used and are commonly available. A stent can be placed using a stent placement balloon catheter, which is similar to a balloon catheter used for PTCA. Typically, the stent is crimped onto the balloon portion of the catheter. The catheter is introduced into the artery of the patient and advanced, usually over a guide wire, until the balloon carrying the stent is adjacent the buildup of material. The balloon is then inflated, thus expanding the stent. The balloon is then deflated and the balloon catheter is removed from the artery. The expanded stent is left to support the stenotic area of the artery.

More recently, it has been discovered that treating the diseased area of the vessel with radiation after angioplasty can further minimize the effect of restenosis. One way to treat the diseased area with radiation is to use a catheter system to advance a wire having a radioactive distal end region to the diseased area. The diseased area may have previously been treated by angioplasty and may have had a stent placed at the area.

In order to deliver a proper amount of radiation to the vessel wall, the radioactive distal end portion of the radiation delivery wire should be centered within the vessel. Centering can be accomplished by using a centering balloon catheter, in which the radiation delivery wire is advanced through a central lumen of the centering balloon catheter. A centering balloon catheter can include multiple balloons. One type of centering balloon catheter includes an inner segmented balloon or several inner balloons and an outer balloon over the inner balloons. This type of multiple balloon catheter can position a central lumen of the catheter within a tortuous or bending vessel such that the radiation delivery wire can be centered within the vessel. Another type of centering balloon catheter includes a “spiral” or “helical” balloon. A spiral or helical balloon catheter includes a balloon that is wrapped around the central lumen. When inflated, the outer surface of the spiral or helical balloon contacts the vessel wall while the inner surface centers the central lumen that will carry the radiation delivery wire.

Some types of centering catheters include one or more inner balloons that perform the centering function and an outer balloon that can perform the compressing function of the angioplasty procedure. The outer balloon can also serve to evacuate blood from the area surrounding the balloon to minimize the attenuation of the radiation delivered by the radiation source wire. In some cases, separate balloons of the multiple balloon catheter should be inflated to different pressures. For instance, the inner balloon may require a higher pressure than the outer balloon because the pressure required to inflate the outer balloon tends to inhibit the inflation of the inner balloon.

Other types of multiple balloon catheters are contemplated for various types of interventional treatment of arterial disease. Multiple balloon catheters can be used for delivery of drugs to a diseased area of an artery. Multiple balloon catheters can include a balloon with individually or separately inflatable lobes or separate balloons arranged on the catheter in proximal and distal relationship to each other. It may also be desirable to provide different pressures to the separate balloons of such a catheter.

Currently, balloon catheters are inflated and deflated using syringes or other inflation devices that may include syringes to deliver the inflation medium through the catheter to the balloon. These devices may include valve systems to control the pressure delivered to the balloons or pressure indicators such as gauges so that the operator can monitor the pressure delivered to the balloon. In multiple balloon catheters it may be necessary to provide more than one inflation device so that the different balloons of the catheter can be inflated to different pressures. However, providing additional components to the catheter system can increase the cost and complexity of the procedure and the time required to complete the procedure.

### SUMMARY OF THE INVENTION

A balloon catheter inflation system embodying the present invention includes a housing defining a chamber for an inflation medium. The housing can be coupled to a balloon catheter that has two balloons. The housing can be coupled to the catheter such that the chamber is in fluid communication with the balloons. The inflation system further includes a valve configuration operatively associated with the chamber to provide a first pressure to one of the balloons and a second pressure to the other balloon by a single inflating action that moves the inflation medium from the chamber to the balloons.

### BRIEF DESCRIPTION OF THE DRAWINGS

The invention is illustrated by way of example, and not limitation, in the figures of the accompanying drawings in which:

FIG. 1 is a perspective view of one embodiment of an inflation device;

FIG. 2 is a plan view of an embodiment of the inflation device of FIG. 1 together with an example of a balloon catheter;

FIGS. 3A through 3E are schematic illustrations of the various stages of an inflation system during use;

FIG. 4 is a schematic illustration of another embodiment of an inflation system;

FIG. 5 is a cross sectional view of an embodiment of a safety relief valve;

FIG. 6 is a cross sectional view of an embodiment of a valve; and

FIG. 7 is a cross sectional view of an embodiment of a pressure regulator.

### DETAILED DESCRIPTION OF THE INVENTION

Various embodiments of an inflation system for balloon catheters are described below. The inflation system of the present invention includes embodiments of inflation devices that can provide different pressure levels to different balloons of the multiple balloon catheter. The different pressure levels can be provided to the multiple balloons by a single inflating action.

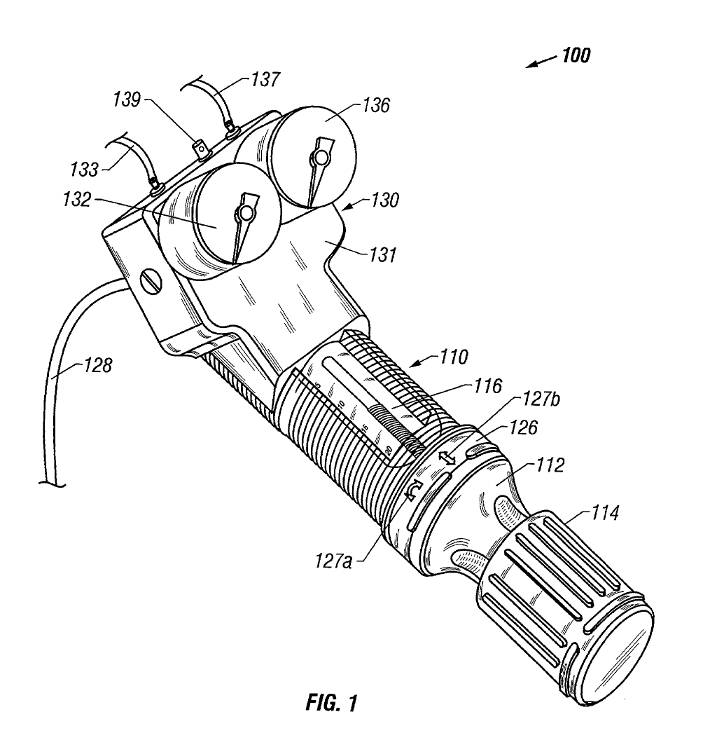

FIG. 1 shows an embodiment of an inflation system of the present invention embodied in inflation device 100. Inflation device 100 is preferably a hand-held and hand operatable device that includes a piston portion 110 and pressure control portion 130.

Piston portion 110 includes a piston body 112, a handle 114 at the end of the piston body 112, and chamber housing 116 at the opposite end of the piston body 112. Chamber housing 116 defines a chamber (not shown) that holds an inflation medium such as a liquid or gas. As will be described in further detail below, the inflation device 100 can be coupled to a balloon catheter such that the inflation medium can be moved through at least one inflation medium pathway to inflate the balloon or multiple balloons of the catheter. A chamber filling tube 128 or a suitable connector can be attached to the inflation device 100 at a convenient location on the piston portion 110 or the pressure control portion 130 to provide a pathway to fill or empty the chamber.

Piston portion 110 can further include a piston mode selection ring 126. Piston mode selection ring 126 cooperates with the piston mechanism (not shown) such that the piston can be moved into and out of the chamber by a sliding motion of the handle 114 when the ring 126 is in a first position or by a ratcheting or turning motion of the handle 114 when the ring 126 is in a second position. The first and second positions of the piston mode selection ring 126 can be indicated by icons 127a and 127b.

Pressure control portion 130 includes a valve housing 131 attached to or unitary with chamber housing 116. A first pressure level indicator 132 and a second pressure level indicator 136 can be provided on the pressure control portion 130. Alternatively, the pressure control portion 130 need not be attached to or unitary with chamber housing 116, but can be provided as a separate unit being connected to the piston portion 110 by a tube or tubes that provide an inflation medium pathway. A first intermediate tube 133 and a second intermediate tube 137 extend from pressure control portion 130 or from housing 131 as shown in FIG. 1. First intermediate tube 133 and second intermediate tube 137 provide a continuation of an inflation medium pathway or pathways from the chamber of the piston portion 110 to the balloon catheter (not shown). Alternatively, the intermediate tubes can be replaced with connectors directly mounted onto the valve housing 131. Valve housing 131 can also include a vent 139. Vent 139 can be used when the inflation medium is a gas that can be vented into the atmosphere. In one embodiment, the inflation medium is sterilized carbon dioxide (CO₂). Carbon dioxide can be vented into the atmosphere in most cases. Also, CO₂ can be absorbed by the blood in the event that some amount of CO₂ escapes into the patient's bloodstream.

Inflation device 100 can be made of a durable plastic such as polycarbonate or metal such as stainless steel. Because inflation device 100 is used in or near a sterile field, inflation device 100 is preferably sterilizeable. It is contemplated, however, that inflation device 100 is a disposable device to be used only once due to the highly critical nature of the catheterization procedures for which the device is used.

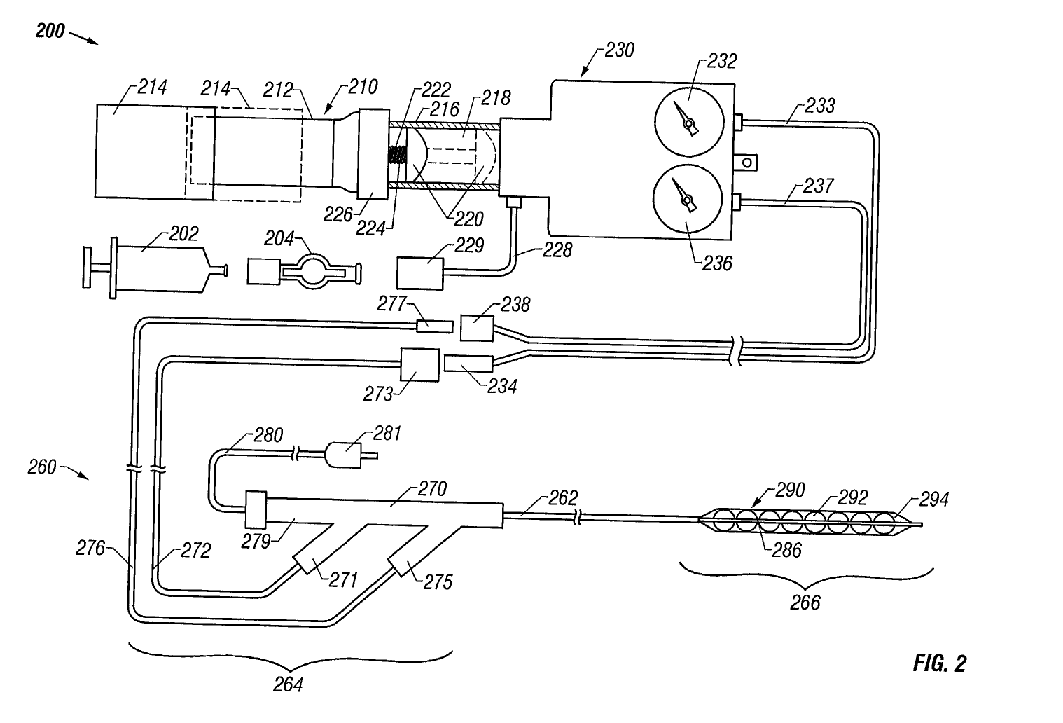

FIG. 2 shows an embodiment of an inflation device 200 together with an example of a balloon catheter 260.

The exemplary balloon catheter 260 shown in FIG. 2 is an example of a radiation source wire centering catheter. Catheter 260 includes a main catheter shaft 262, a proximal end region 264 at one end of the main catheter shaft 262 and a distal end region 266 at the opposite end of the main catheter shaft 262.

The distal end region 266 includes balloon assembly 290. Balloon assembly 290 of the exemplary balloon catheter 260 of FIG. 2, includes a plurality of first balloons 292 and a second balloon 294. The inner (or first) balloons 292 perform the centering function.

The plurality of first balloons 292 is arranged to provide a series of adjacent inner balloons (or multiple lobes or an inner balloon) that carry radiation source wire lumen 286, which extends centrally through the series of inner balloons 292. The plurality of first balloons 292 can be a series of separate balloons or one segmented balloon that provides lobes forming a series of peaks and valleys such that the balloon assembly 290 can be inserted into a tortuous or bending blood vessel while maintaining the radiation source wire lumen 286 positioned centrally within the blood vessel. Second balloon 294 is an oblong outer balloon that covers the plurality of first balloons 292. Second balloon 294, when inflated, evacuates blood from the area surrounding balloon assembly 290 in order to lessen the attenuating effects of the blood in the artery so that the radiation from the source wire can be delivered evenly to the wall of the diseased portion of the artery.

While embodiments of the invention are described with reference to a first balloon and a second balloon of a catheter having two or multiple balloons, it should be understood that the invention can be applied to an inflation device that can inflate catheters having more than two balloons that can be inflated to two or more different pressures.

Referring to FIG. 2, in order to inflate the first balloon 292 (i.e. the series of inner balloons in the example of FIG. 2), the pressure of the inflation medium delivered to first balloon 292 must be higher than the pressure of the inflation medium delivered to second (or outer) balloon 294. This is because, in this example, the inner balloons must overcome the pressure inside of the outer balloon.

A catheter junction 270 is provided at the proximal end region 264. Catheter junction includes a first arm 271, a second arm 275, and a third arm 279. Extending from the first arm 271 is a first proximal tube 272, and extending from the second arm 275 is a second proximal tube 276. First proximal tube 272 and second proximal tube 276 extend through catheter junction 270 and are in fluid communication with respective inflation lumens defined within the main catheter shaft 262. The first and second proximal tubes, together with their respective inflation lumens, provide separate inflation medium pathways to the multiple balloon assembly 290 at the proximal end region 266 of the balloon catheter 260.

A third proximal tube 280 extends from the third arm 279 of the catheter junction 270. Third proximal tube 280 is in fluid communication with a source wire lumen that can accept a radioisotope-tipped source wire. The third proximal tube 280 can include or accept a reinforcing stylet (not shown) to assist the introduction of the catheter into to the arterial system of the patient.

A guide wire (not shown) can be inserted through a short lumen distal to the balloon during the initial preparation for the insertion of the catheter. Once the guide wire is extended past the distal end region 266 of the catheter 260, the guide wire can be introduced into the arterial system of the patient and advanced so that the distal tip of the guide wire is ultimately positioned past the target region of the artery to be treated. Next, the balloon catheter 260 (with the balloon assembly 290 still deflated) is advanced into the artery of the patient to position the distal end region 266 including the balloon assembly 290 adjacent the target area to be treated.

The stylet is then withdrawn from the proximal tube 280. A radiation source wire (not shown) is then inserted through the third proximal tube 280. Third proximal tube 280 can be provided with an afterloader connector 281 for quick connection to an afterloader, which is a device that automatically advances a radiation source wire through such a balloon catheter during radiation therapy to treat restenosis.

To provide a connection between the catheter 260 and the inflation device 200, a first proximal tube connector 273 and the second proximal tube connector 277 are provided at the respective ends of the proximal tubes 272 and 276. As illustrated schematically in FIG. 2, connectors 273 and 277 have different sexes to correspond to respective oppositely-sexed connectors on the inflation device 200.

Proximal tube connectors 273 and 277 can also be color-coded to further reduce the possibility of improper connection of the first and second proximal tubes 272 and 276 to the inflation device. Since first and second proximal tubes 272 and 276 are in fluid communication with first balloon 292 and second balloon 294, respectively, of the balloon assembly 290, the oppositely sexed connectors provide an automatic connection to the corresponding inflation medium pathway that provides the proper pressure level to each balloon.

FIG. 2 shows an embodiment of an inflation device 200 that can be used with balloon catheter 260 or another multiple balloon catheter. Inflation device 200 can provide two different pressure levels to separate balloons of the multiple balloon catheter such as exemplary catheter 260 of FIG. 2.

Inflation device 200 includes a piston portion 210 and pressure control portion 230. Piston portion 210 includes a piston body 212. In the embodiment shown in FIG. 2, piston body 212 is an elongated cylindrical member that is hollow to accommodate a piston shaft as described below.

Handle 214 is associated with piston body 212 such that handle 214 is slidable longitudinally with respect to piston body 212. A handle 214 is also rotatable about the coaxial longitudinal axes of the piston body 212 and the handle 214.

The piston portion 210 further includes a chamber housing 216 defining the chamber 218 that carries the inflation medium (not shown). A piston head 220 is coaxial with and slidably received within the chamber 218. The piston head 220 is mounted to a piston shaft 222, which in turn is attached to the handle 214 such that manipulation of the handle ultimately slides the piston head 220 longitudinally within the chamber 218.

When the handle 214 and the piston head 220 are positioned as shown by the solid lines of FIG. 2, the chamber 218 can accommodate a predetermined volume of an inflation medium that is to be moved from the chamber through at least one inflation medium pathway, further through the catheter, and ultimately to fill at least one of the balloons at the distal end region 266 of the catheter 260. The broken lines illustrating the handle 214 and piston 220 in FIG. 2 show the second position of the handle 214 and piston 220 in which the inflation medium has been moved from the chamber 218 through the pressure control portion 230 and further through the inflation medium pathways into the balloons of the catheter 260. In this manner, the handle 214 and piston head 220 act as a syringe to push the inflation medium into the balloons to be inflated with a single inflating action.

In the embodiment of the inflation device 200 of FIG. 2, a piston mode selection ring 226 can be provided on the piston portion 210. Piston mode selection ring 226 is coaxial and surrounds the piston body 212. The piston mode selection ring 226 can include a mechanism which is engagable and disengagable with the piston shaft 222 such that when the selection ring 226 is in a first position, the handle 214, piston head 220, and piston shaft 222 are slidable longitudinally with respect to the piston body 212 and the chamber housing 216. In the first position, the selection ring 226 is disengaged from the piston shaft 222. In a second position, the selection ring 226 can be engaged with the piston shaft 222 such that the longitudinal movement of the piston head 222 within the chamber 218 can be more precisely controlled. For instance, when the selection ring 226 is in the second position, rotation or ratcheting of the handle 214 is required to move the piston head 220 longitudinally within the chamber 218.

To provide more precise control of the movement of the piston head 220, the piston mode selection ring 226 includes a mechanism that cooperates with helical threads 224 of the piston shaft 222. Alternately, rather than helical threads 224, a series of teeth can be provided on shaft 222 so that a ratcheting action will move the piston head 220 within the chamber 218.

FIG. 2 also shows a syringe 202 and a stopcock 204 that can be associated with the inflation device 200. Syringe 202 can be used to transfer an inflation medium such as carbon dioxide gas from a tank, for example, that is outside of the sterile field to the inflation device 200 in preparation for inflating the balloons of the catheter. Stopcock 204 can be provided to open and close the fluid pathway between the syringe and the chamber of the inflation device 200. Syringe 202 can be connected via stopcock 204 to the connector 229 on the chamber filling tube 228. Alternatively, the connector 229 can be directly mounted to the inflation device 200 without the intermediate chamber filling tube 228.

The pressure control portion 230 of the inflation device 200 can be coupled to or made unitary with the piston portion 210. Alternatively, the pressure control portion 230 can be a separate unit coupled between the inflation medium chamber 218 and the catheter 260.

The pressure control portion 230 carries the valve configuration or pressure regulator configuration that provides the different pressure levels to the separate balloons of the catheter. Pressure control portion 230 can include a first pressure level indicator 232 and a second pressure level indicator 236.

A first intermediate tube 233 and a second intermediate tube 237 can extend from the pressure control portion 230 to provide portions of the inflation medium pathways between the chamber 218 and the balloon catheter 260. A first tube connector 234 is provided at the end of the first intermediate tube 233 and the second tube connector 238 is provided at the end of the second intermediate tube 237. The first tube connector 234 cooperates with the first proximal tube connector 273 of the catheter 260, and the second tube connector 238 cooperates with the second proximal tube connector 277 of the catheter. These connectors can be of a variety of types commonly used in the medical device field, such as Luer connectors, twist lock connectors, or threaded male and female connectors. Alternatively, connectors 234 and 238 can be provided on the inflation device 200 without the intermediate tubes 233 and 237.

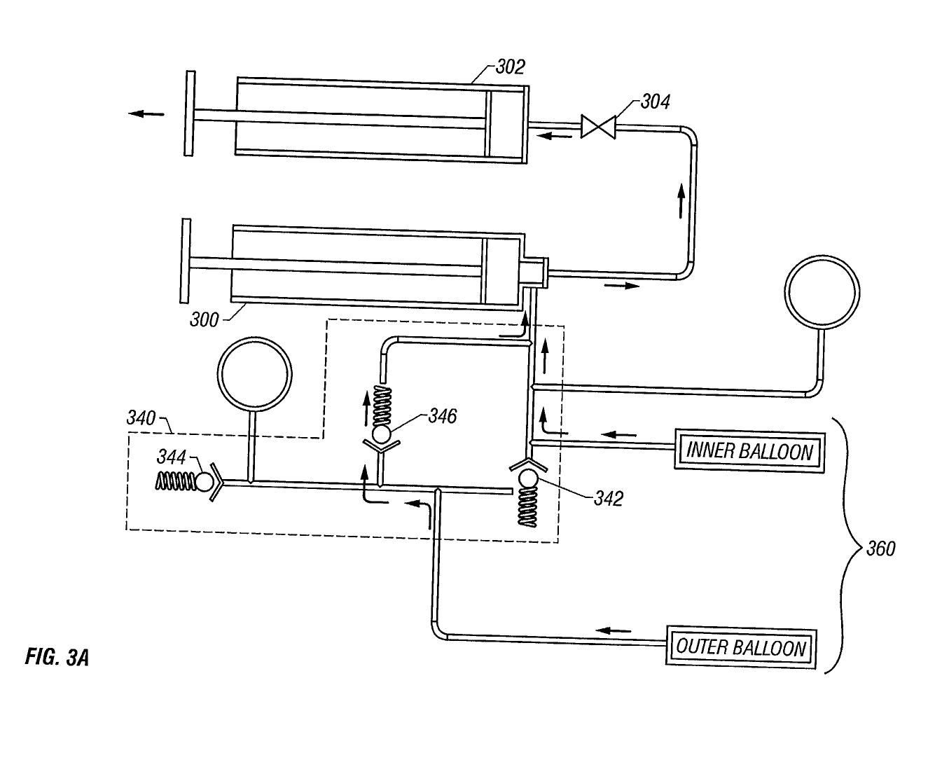

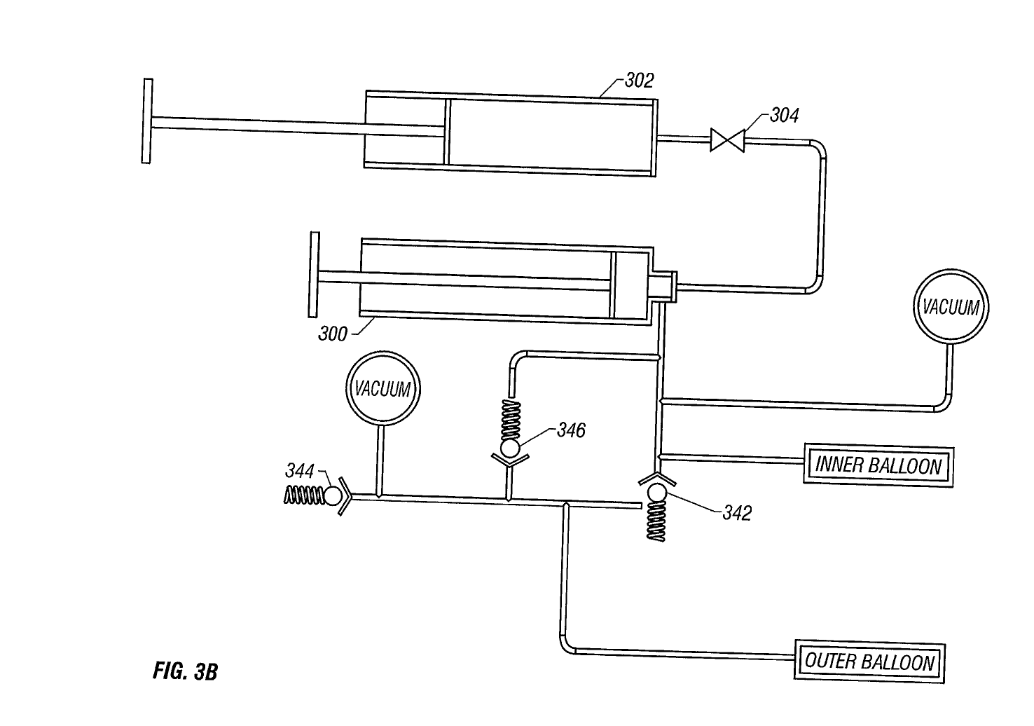

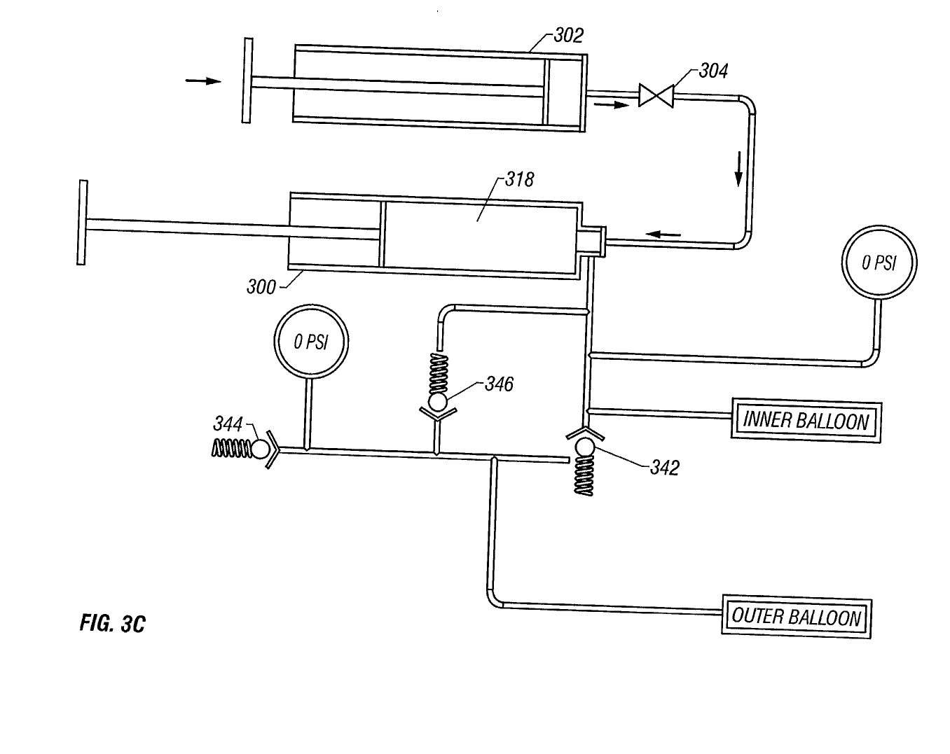

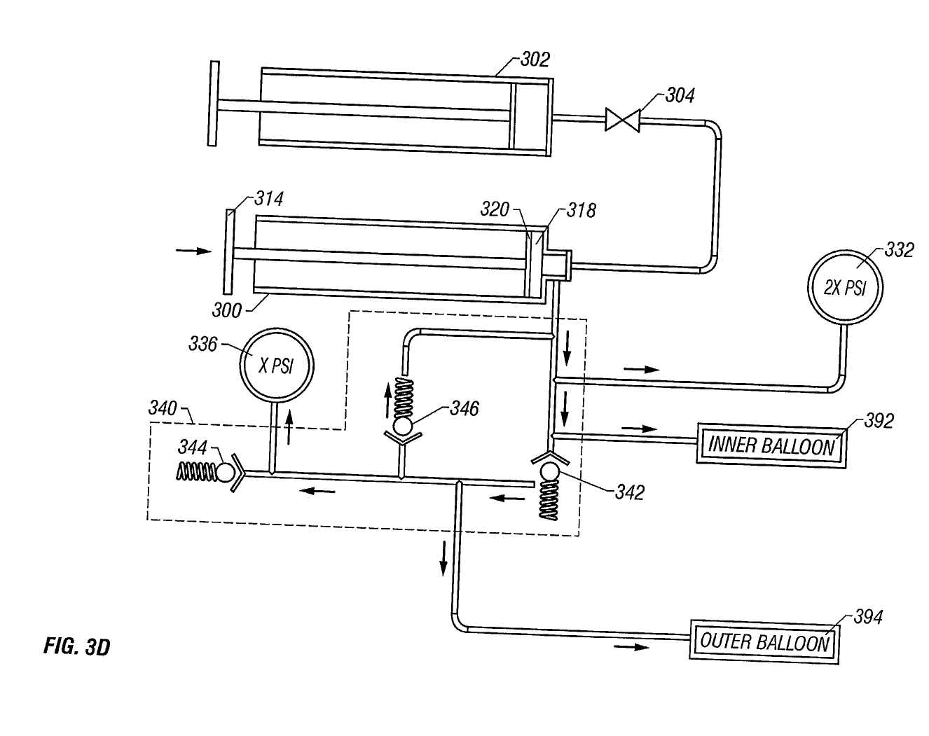

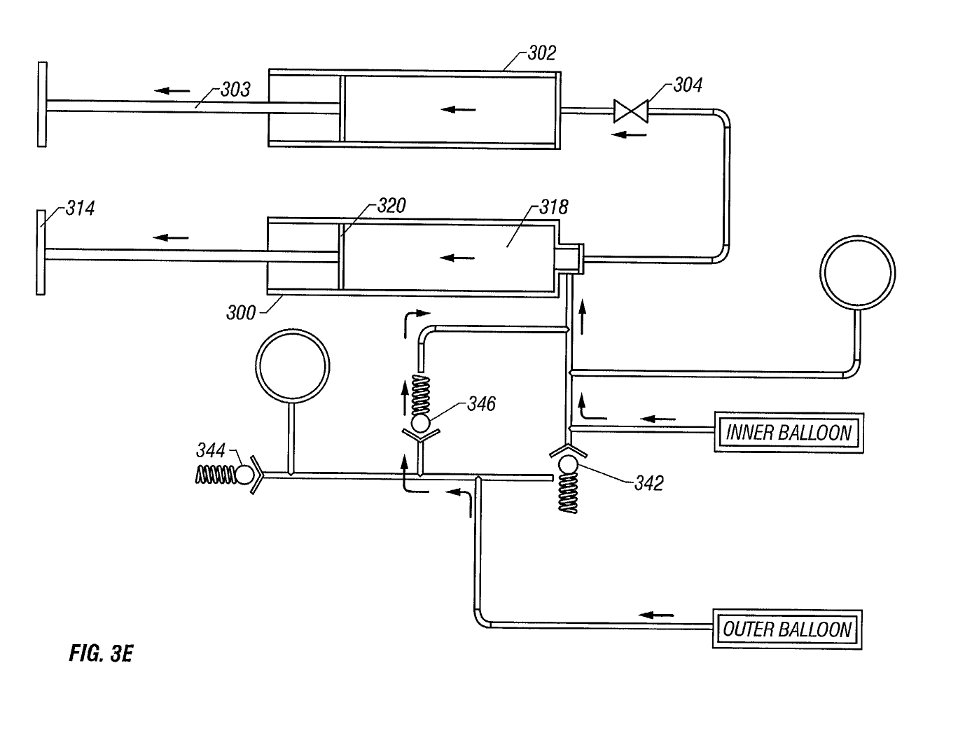

FIGS. 3A through 3E show simplified schematic illustrations of the catheter inflation system of the present invention together with a balloon catheter having an inner and an outer balloon. The figures illustrate the various stages of the inflation device and valve configuration during inflation and deflation of the balloons of the catheter.

FIG. 3A shows the inflation device 300 connected to a radiation centering catheter 360 and a syringe 302. The entire system is aspirated of air by pulling back on the syringe 302. The one way stopcock 304 is then closed to seal off the system. The arrows of FIG. 3A illustrate the direction of air flow.

A valve configuration 340 is represented in FIG. 3A by the portion of the diagram within dashed lines. Valve configuration 340 includes the components of the system or inflation device 300 that would be located in the pressure control portion of the inflation device 300. Valve configuration 340 includes a first pressure relief valve 342, a second pressure relief valve 344, and check valve 346 arranged in a circuit that provides a different pressure level to each balloon by a single inflating action that moves the inflation medium from the chamber to the balloons substantially simultaneously, as described in further detail below.

That check valve 346 allows a free flow of air or inflation medium when a vacuum is applied via the inflation device or the syringe. Both balloons can thus be substantially simultaneously deflated.

The actions illustrated by the schematic of FIG. 3A include opening the one way stopcock 304, pulling back on the plunger of the syringe 302, and closing the one way stopcock 304. At this point, the system is under vacuum, having been aspirated of air by the creation of vacuum through the action of pulling back on the syringe.

FIG. 3B illustrates the inflation system in which the syringe 302 has been filled with an inflation medium. An inflation medium can be a liquid or a gas. In the case of a gaseous inflation medium, the gas should be one that is readily absorbed by the body fluid (i.e. blood). One exemplary gas that can be used is sterile CO₂ gas. The gas may itself be radioactive and can have a treatment function as well as being an inflation medium. In the embodiment in which the inflation medium is a liquid, an example of a suitable liquid is the liquid contrast that is typically used to inflate balloon catheters such as those used for angioplasty. Also, liquids or slurries carrying radioactive isotopes are contemplated as being suitable inflation media. An example of a radioactive isotope is phosphorus 32 (³²P).

In FIG. 3B, the one way stopcock 304 remains closed so that the following actions can be performed. The actions illustrated by the schematic of FIG. 3B include disconnecting the syringe 302 from the one way stopcock 304, filling the syringe 304 with an inflation medium, and reconnecting the syringe 302 to the one way stopcock 304.

FIG. 3C illustrates the inflation system in which the chamber 318 of the inflation device 300 is filled with the inflation medium from the syringe 302. The actions illustrated by FIG. 3C include opening the one way stopcock 304, injecting the inflation medium into the inflation device 300 by pushing the plunger 303 of the syringe 302, and closing the one-way stopcock 304.

FIG. 3D illustrates the inflation system pressurized by the inflation device 300. At this stage, both the first balloon 392 (or inner balloons) and the second balloon 394 (or outer balloon) are inflated. In FIG. 3D, the handle 314 and the piston head 320 have been moved forwardly to compress the volume of the inflation within the chamber 318. The one way stopcock 304 remains closed to prevent the inflation medium from escaping into the syringe 302.

In the embodiment illustrated in FIG. 3D, the first pressure level indicator 332 and the second pressure level indicator 336 indicate that the pressure of the first balloon 392 is about twice that of the second balloon 394. For example, in one embodiment the inner or first balloon 392 can have a pressure level of about 30 psi (approximately two atmospheres) and the outer or second balloon 394 can have a pressure level of about 15 psi (approximately one atmosphere).

The movement of the piston handle 314 and the piston head 320, in the embodiment of FIG. 3D, is one embodiment of the single inflating action that moves the inflation medium from the chamber 318 to the balloons 392 and 394. The valve configuration 340 includes the first pressure relief valve 342, the second pressure relief valve 344, and the check valve 346 arranged in a circuit to provide a pressure level to the first balloon 392 that is approximately twice the pressure level provided to the second balloon 394.

In the embodiment of FIG. 3D, the first and second pressure relief valves 342 and 344 can each have the same cracking pressure. For example, both the first pressure relief valve 342 and the second pressure relief valve 344 can be set at a one atmosphere cracking pressure. Because of the arrangement of the relief valves and the check valve 346 in the circuit; and in particular, because the first pressure relief valve 342 has one atmosphere of back pressure acting on it, first pressure relief valve 342 provides a pressure level to the first balloon 392 that is approximately twice that of the pressure level of the second balloon 394. The check valve 346 can be set to a lower cracking pressure, for example, five psi.

The first and second pressure relief valves 342 and 344 provide a maximum pressure to each of their respective balloons as a safety feature. The balloons can be designed to withstand a maximum pressure before over inflating or bursting and the pressure relief valves can be selected or calibrated to release pressure well within the range that the balloons can withstand.

FIG. 3E illustrates the inflation system being aspirated or purged of the inflation medium. The actions illustrated by the schematic of FIG. 3E include pulling back the handle 314 and thus the piston head 320 of the inflation device 300 to deflate the balloons and create a vacuum within the chamber 318 such that the inflation medium is moved back into the chamber 318 of the inflation device 300. Another action illustrated in FIG. 3E is opening the one way stopcock 304 and pulling back the plunger 303 of the syringe 302 to move the inflation medium from the chamber 318 of the inflation device 300 into the syringe 302.

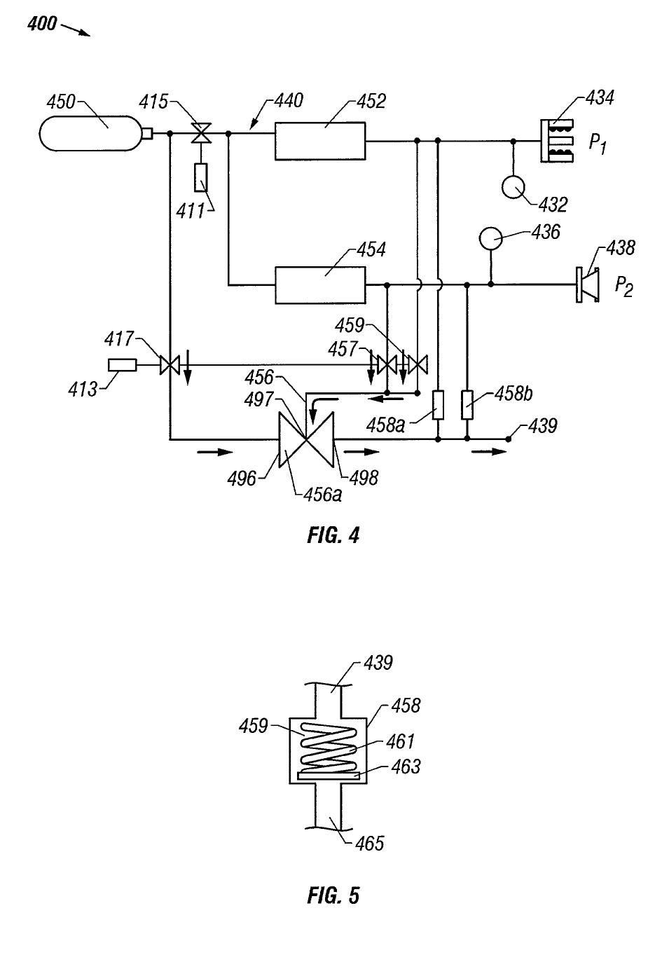

FIG. 4 shows a schematic illustration of another embodiment of an inflation system in which inflation device 400 includes a self-contained inflation medium source 450. In one embodiment, the inflation medium source 450 can be housed within the inflation device 400. If the inflation medium is a gas, such as sterilized CO₂, the inflation medium source 450 can be a pressurized vessel, for example. If the inflation medium is a liquid, such as radiopaque contrast or a liquid carrying a radioactive isotope, the inflation medium source 450 can include a cylinder or canister with a spring-loaded or manually operated piston, for example. Another embodiment of the inflation medium source 450 can include a refillable reservoir. The inflation medium source 450 can be included in the inflation device or can be a separate unit that is coupleable to be in fluid communication with the inflation device.

FIG. 4 is a schematic diagram of a valve configuration 440 that provides two different pressure levels of an inflation medium to a balloon catheter (not shown) and ultimately to separate balloons of the balloon catheter such that the balloons can each be inflated to different pressures substantially simultaneously and by a single inflation action. The valve configuration 440 shown in FIG. 4 includes a first pressure regulator 452 and a second pressure regulator 454 that provide the separate pressure levels through an inflation medium pathway to a first connector 434 and a second connector 438, respectively. First connector 434 and second connector 438 are adapted to be coupled to corresponding connectors or coupling points of a balloon catheter. A first pressure level indicator 432 can be provided to indicate the first pressure level P1, and a second pressure level indicator 436 can be provided to indicate a second pressure level P2.

A selectively actuated valve 415 can be provided between the inflation medium source 450 and the first and second pressure regulators 452 and 454. Inflation valve 415 can be actuated by a valve actuator 411. In this embodiment, a single inflation action is the actuation of the valve actuator 411 which opens valve 415 to allow the pressurized or spring loaded inflation medium which inflation medium source 450 to flow to first and second pressure regulators 452 and 454. The pressure regulators 452 and 454 can be individually selected to provide different pressures to the first and second connectors 434 and 438, and ultimately to two separate balloons (not shown) of a dual balloon catheter coupled to connectors 434 and 438. When the valve actuator 411 is released, i.e., is not causing valve 415 to be open to allow flow of the inflation medium, the valve 415 is closed to provide a closed system which keeps the balloons of the catheter inflated.

Safety relief valves 458a and 458b can be provided in the circuit between the first and second pressure regulators and the first and second connectors 434 and 438. Safety relief valves 458a and 458b allow excess pressurized gas or inflation medium to be vented through vent 439. Alternatively, valves 458a and 458b can be used in place of pressure regulators 452 and 454 to provide the pressures P₁ and P₂ at connectors 434 and 438. Also, in the case of the inflation medium being a liquid, vent 439 can provide a collection point for the liquid purged or drawn from the system. For instance, a Luer-type or similar fitting can be provided at vent 439 for coupling to a syringe or other collection device. The syringe or other suction device can also provide a backup Venturi system to deflate the balloons.

Selectively actuated valve 417 can be provided in the circuit and can be operated by a valve actuator 413 to deflate the balloons of the catheter. Valve 417 works in conjunction with valves 457 and 459, Venturi vacuum line 456, and Venturi tube 456a. In this embodiment, the pressurized line of the valve configuration 440 can be used to create a vacuum for balloon deflation. In other embodiments, balloon deflation can be provided by systems such as a Venturi system, a turbine which powers a vacuum pump, opposed reservoirs (in which one is filled, forcing the other to also fill), and the like.

In an embodiment that uses a pressurize gas as the inflation medium, FIG. 4 shows how the system can be deflated by using the system of valves and the pressurized gas source and a Venturi tube. Valve actuator 413 can open valves 417, 457 and 419 simultaneously. When valves 417, 457 and 459 are open, the gas flows in the direction of the arrows of FIG. 4. Venturi tube 456a has two inlets 496 and 497. Opening valve 417 allows a pressurized flow of gas into inlet 496 and through Venturi tube 456a and further through outlet 498. This pressurized gas flow causes a Venturi effect which creates a vacuum at inlet 497. Vacuum line 456 is coupled to inlet 497. The vacuum effect draws the gas in the direction of the arrows next to valve 457 and 459 which are in fluid communication with the inflation medium pathways that lead to the balloons of the catheters. Thus, the balloons are deflated as valves 457 and 459 are opened by the actuation of valve actuator 413 and the vacuum effect created by the pressurized flow of gas through the Venturi tube 456a and ultimately out through vent 439.

Other embodiments of the valve configuration 440 can include color-coded pressure fittings or connectors, male and female Luer connectors to ensure that the correct inflation pathway is connected to the proper catheter inflation tube, and an additional vacuum port to provide an option in the event the deflation system of FIG. 4 fails. Flow valves can be included to provide a desired flow rate. Alternatively, the diameters of the inflation medium pathways can be selected to provide the desired flow rate.

In a single balloon catheter, different pressure levels can be provided at different times. In another example, two or more separate balloons of a balloon catheter can be inflated to different pressure levels substantially simultaneously through a single inflating action.

FIG. 5 shows an embodiment of a safety relief valve 458 that can be used in the various embodiments of the inflation devices described herein. Safety relief valve 458 can be provided in a cavity 459 that is molded into the fluid pathway. An inflation medium under pressure can travel from the input 455 side of the safety valve 458 through the valve to the vent 439. A spring 461 biases a disk 463 to seal the inflation medium pathway until the biasing force of the spring 461 is overcome by the pressure of the inflation medium.

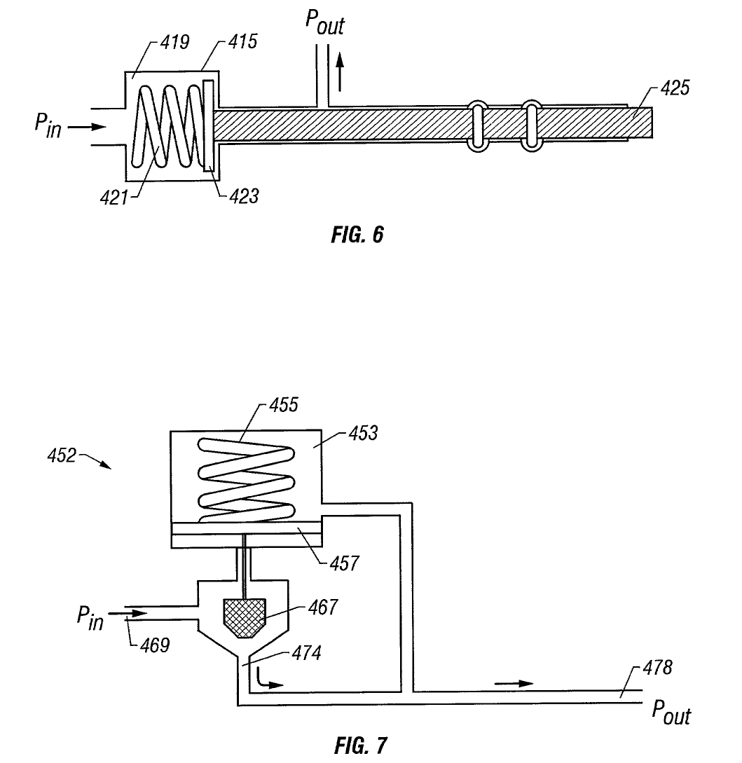

FIG. 6 shows an embodiment of a selectively actuated valve 415. The embodiment of FIG. 6 can be used for valves 417, 457 and 459 as well. Inflation valve 415 can include a cavity 419 that is molded into the fluid pathway. A spring 421 biases a disk 423 that is connected to a rod 425 to keep the valve 415 closed until the rod 425 is manually pressed to overcome the biasing force of the spring 421. When the rod 425 is pressed, the inflation valve 415 is open, and the inflation medium can travel through the valve 415 in the direction of the arrows of FIG. 6.

FIG. 7 shows an example of a pressure regulator 452. Pressure regulator 452 includes a cavity 453 molded into the inflation medium pathway. A spring 455 provides a biasing force against a diaphragm or piston 457, which in turn holds a stopper 467 in a closed position until the inflation medium pressure at the input 469 overcomes the biasing force of the spring 455 by providing fluid pressure against piston 457 in the opposite direction. Thus, stopper 467 is moved away from an inner opening 474 in the inflation medium pathway such that the inflation medium can travel under pressure to the output 478 of the pathway in the direction of the arrows of FIG. 7.

The various embodiments of an inflation device described herein can provide substantially simultaneous and different pressure levels to separate balloons of a multiple balloon catheter. Embodiments of inflation devices of the present invention are particularly useful to inflate radiation centering balloon catheters that include an inner balloon or set of balloons that provide the centering function of the catheter and also an outer balloon covering the inner balloon or balloons that serves to evacuate blood away from the interior of the vessel adjacent the balloon assembly. Typically, a higher pressure is required to inflate the inner balloons of such a balloon catheter because when the outer balloon is inflated, the pressure within the outer balloon tends to deflate the inner balloons unless the inner balloons are inflated to a pressure that can overcome the pressure inside the outer balloon.

The various embodiments of the present invention can also reduce the complexity of a multiple balloon catheter inflation procedure. A relatively simple hand-held inflation device that inflates at least two separate balloons to different pressures by a single inflating action can greatly reduce the time required to perform the inflation procedure and thus, can reduce that overall catheterization or radiation treatment procedure that the patient experiences. Also, a decrease in the number of components required to accomplish the multiple balloon inflation to different pressures can lower the risk of errors associated with failure of a component or operator error due to confusion.

What is claimed is:

1. A balloon catheter inflation system, comprising:
a housing to define a chamber for an inflation medium, the housing coupleable to a balloon catheter that has at least two balloons, the housing being coupleable to the catheter such that the chamber is in fluid communication with the balloons; and
a valve configuration operatively associated with the chamber to provide a first pressure to one of the balloons and a second pressure to the other balloon by a single inflating action that moves the inflation medium from the chamber to the balloons.

2. The system of claim 1 wherein the valve configuration includes at least one pressure relief valve.

3. The system of claim 2 wherein the valve configuration includes a first pressure relief valve to provide the first pressure and a second pressure relief valve to provide the second pressure.

4. The system of claim 3 wherein the first pressure relief valve and the second pressure relief valve both have about the same cracking pressure and are arranged in a circuit such that the first pressure is about twice that of the second pressure.

5. The system of claim 3 wherein the inflation medium travels to both balloons substantially simultaneously during the single inflating action.

6. The system of claim 3 further comprising a first inflation medium pathway between the chamber and a first balloon and a second inflation medium pathway between the chamber and a second balloon, wherein the first pressure relief valve is associate with the first pathway and the second pressure relief valve is associated with the second pathway.

7. The system of claim 1 wherein the valve configuration includes at least one pressure regulator.

8. The system of claim 7 wherein the valve configuration includes a first pressure regulator to provide the first pressure and a second pressure regulator to provide the second pressure.

9. The system of claim 8 wherein the first pressure regulator and the second pressure regulator are arranged in a circuit such that the first pressure is about twice that of the second pressure.

10. The system of claim 8 wherein the inflation medium travels to both balloons substantially simultaneously during the single inflating action.

11. The system of claim 8 further comprising a first inflation medium pathway between the chamber and a first balloon and a second inflation medium pathway between the chamber and a second balloon, wherein the first pressure regulator is associated with the first pathway and the second pressure regulator is associated with the second pathway.

12. The system of claim 1 wherein the housing includes a piston that is slidable within the chamber, and wherein the piston provides the single inflating action which evacuates the inflation medium from the chamber.

13. The system of claim 1 wherein the single inflating action is manually operatable.

14. The system of claim 1 wherein the valve configuration is integral with the housing.

15. A balloon catheter inflation system, comprising:
a housing to define a chamber for an inflation medium, the housing coupleable to a balloon catheter that has at least two balloons, the housing being coupleable to the catheter such that the chamber is in fluid communication with the balloons; and
means for providing a first pressure to one of the balloons and a second pressure to the other balloon by a single inflating action that moves the inflation medium from the chamber to the balloons.

16. The system of claim 15 wherein means for providing a first pressure to one of the balloons and a second pressure to the other balloon by a single inflating action includes a valve configuration having a first pressure relief valve to provide the first pressure and a second pressure relief valve to provide the second pressure.

17. The system of claim 16 wherein the first pressure relief valve and the second pressure relief valve both have about the same cracking pressure and are arranged in a circuit such that the first pressure is about twice that of the second pressure.

18. The system of claim 15 wherein the means for providing a first pressure to one of the balloons and a second pressure to the other balloon by a single inflating action includes at least one pressure regulator.

19. The system of claim 18 wherein the means for providing a first pressure to one of the balloons and a second pressure to the other balloon by a single inflating action includes a first pressure regulator to provide the first pressure and a second pressure regulator to provide the second pressure.

20. The system of claim 15 further comprising means for providing the first pressure at a level that is about twice that of the second pressure.

21. The system of claim 15 further comprising means for evacuating the inflation medium from the chamber.

22. The system of claim 15 wherein the means for providing a first pressure to one of the balloons and a second pressure to the other balloon by a single inflating action is integral with the housing.

23. The system of claim 15 wherein the means for providing a first pressure to one of the balloons and a second pressure to the other balloon by a single inflating action is manually operatable.

24. The system of claim 15 wherein the means for providing a first pressure to one of the balloons and a second pressure to the other of the balloons by a single inflating action comprises two syringes, each connectable by oppositely sexed connectors to the balloon catheter such that each syringe is in fluid communication with one of the two separate balloons.

25. The system of claim 15 further comprising means for moving the inflation medium to both balloons substantially simultaneously.

26. A method of inflating the balloons of a multiple balloon catheter, the method comprising:
coupling an inflation device to a balloon catheter that has at least two balloons, the inflation device including a housing defining a chamber for an inflation medium, the housing being coupleable to the catheter such that the chamber is in fluid communication with the balloons; and
providing a first pressure to one of the balloons and a second pressure to the other balloon by a single inflating action that moves the inflation medium from the chamber to the balloons.

27. The method of claim 26 further comprising providing the first pressure at a level that is about twice that of the second pressure.

28. The method of claim 26 further comprising evacuating the inflation medium from the chamber.

29. The method of claim 26 further comprising moving the inflation medium to both balloons substantially simultaneously.
